(19) United States
(12) Patent Application Publication
(10) Pub. No.: US 2019/0200903 A1
(43) Pub. Date: Jul. 4, 2019

(54) INTRAORAL VIZUALIZATION SYSTEMS AND METHODS FOR USING SAME

(71) Applicant: Kare Mobile Inc., Louisville, KY (US)

(72) Inventor: Kwane Watson, Louisville, KY (US)

(21) Appl. No.: 16/195,849

(22) Filed: Nov. 19, 2018

Related U.S. Application Data
(60) Provisional application No. 62/588,349, filed on Nov. 19, 2017.

(51) Int. Cl. A61B 5/1178; H04N 5/225; G16H 80/00; A61B 1/24; A61B 5/00; A61C 17/00; A61C 5/50; A61C 5/30; A61B 1/045; H04N 13/204

(52) U.S. Cl. CPC A61B 5/1178; G06Q 40/08; G16H 80/00; A61B 1/24; A61B 5/0013; A61B 5/0088; A61C 17/00; A61C 5/50; A61C 5/30; A61B 1/045; A61B 5/7465; A61B 5/7435; H04N 13/204; H04N 2005/2255; H04N 5/2256

(57) ABSTRACT

Disclosed herein are devices and systems for intraoral visualization. In one aspect, an intraoral visualization system includes at least one intraoral visualization device, each intraoral visualization device comprising: a casing comprising a base and a head; at least one imaging device contained within the head, the at least one imaging device configured to capture video data; a plurality of lighting units contained within the head, each lighting unit comprising a discrete lighting source; a communications module; a power module; and at least one switch in operative communication with and configured to control operation of at least one of: the at least one imaging device or the plurality of lighting units. Also disclosed herein are methods for using the disclosed intraoral visualization devices and systems.

### CROSS REFERENCE TO RELATED APPLICATIONS

This application claims the benefit of priority to U.S. Provisional Patent Application Ser. No. 62/588,349, filed Nov. 19, 2017, which is hereby incorporated herein by reference in its entirety.

### FIELD OF INVENTION

The present disclosure generally relates to intraoral visualization devices and systems and methods for using same. More specifically, various embodiments of the present disclosure relate to imaging and visualization systems used in a dental environment.

### BACKGROUND OF THE INVENTION

There is need to provide access to dental care for individuals in remote areas that may not have access to dental providers. Tele-dentistry may provide a possible solution for providing dental care to these individuals. However, there remains a need for imaging systems and devices that can allow for the remote delivery of dental care.

### SUMMARY OF THE INVENTION

In accordance with the purposes of the invention, as embodied and broadly described herein, the invention, in one aspect, relates to methods, devices, and systems for intraoral visualization.

In another aspect, an intraoral visualization system with integrated peripherals may be provided. This brief overview is provided to introduce a selection of concepts in a simplified form that are further described below in the detailed description. This brief overview is not intended to identify key features or essential features of the claimed subject matter. Nor is this brief overview intended to be used to limit the claimed subject matter's scope. In some embodiments, the integrated peripherals may be provided without intraoral visualization device, but configured to be adapted into an intraoral visualization device to form a system consistent with embodiments of the present disclosure. The system may be integrated with a plurality of peripheral devices such as, for example, but not limited to, at least one imaging device configured to capture video data, a communications module, lighting units, a power module, and a controller. The controller can be configured to control operation of the integrated peripheral devices based on, for example, but not limited to, instructions received via the communications module and a location of the intraoral visualization device. In another aspect, the invention may allow the user to control one or more intraoral visualization device(s) by using a wireless device running application software (an “app”).

In further aspects, the invention also relates to methods for using the disclosed intraoral visualization devices and systems.

Both the foregoing brief overview and the following detailed description provide examples and are explanatory only. Accordingly, the foregoing brief overview and the following detailed description should not be considered to be restrictive. Additional aspects of the invention will be set forth in part in the detailed description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this specification, illustrate several aspects of the invention and together with the description, serve to explain the principles of the invention. The drawings may contain representations of various trademarks and copyrights owned by Applicant. In addition, the drawings may contain other marks owned by third parties and are being used for illustrative purposes only. All rights to various trademarks and copyrights represented herein, except those belonging to their respective owners, are vested in and the property of Applicant. Applicant retain and reserve all rights in their trademarks and copyrights included herein, and grant permission to reproduce the material only in connection with reproduction of the granted patent and for no other purpose. Furthermore, the drawings may contain text or captions that may explain certain embodiments of the present disclosure. This text is included for illustrative, non-limiting, explanatory purposes of certain embodiments detailed in the present disclosure.

FIG. 1 shows a depiction of an intraoral visualization system in accordance with an exemplary embodiment of the present invention.

FIG. 2 shows various views depicting of an intraoral visualization device in accordance with an exemplary embodiment of the present invention.

FIG. 3 shows a diagram depicting a head of an intraoral visualization device in accordance with exemplary embodiments of the present invention

FIG. 4 illustrates a block diagram of an operating environment in accordance with an exemplary embodiment of the present invention.

FIG. 5 shows screen shots of a user interface for a tele-dentistry platform in accordance with an exemplary embodiment of the present invention.

FIG. 6 show screen shots of a user interface for a tele-dentistry platform in accordance with an exemplary embodiment of the present invention.

FIG. 7 shows screen shots of a user interface for a tele-dentistry platform in accordance with an exemplary embodiment of the present invention.

FIG. 8 shows screen shots of a user interface for a tele-dentistry platform in accordance with an exemplary embodiment of the present invention.

FIG. 9 shows screen shots of a user interface for a tele-dentistry platform in accordance with an exemplary embodiment of the present invention.

FIG. 10 shows a method for using the disclosed intraoral visualization devices and systems in accordance with another exemplary embodiment of the present invention.

FIG. 11 shows a diagram of a system including a computing device for enabling operation of the disclosed intraoral visualization devices in accordance with another exemplary embodiment of the present invention.

### DETAILED DESCRIPTION OF THE INVENTION

The present invention can be understood more readily by reference to the following detailed description of the invention and the Examples included therein. As a preliminary matter, it will readily be understood by one having ordinary skill in the relevant art that the present disclosure has broad utility and application

Before the present articles, systems, devices, and/or methods are disclosed and described, it is to be understood that they are not limited to specific manufacturing methods unless otherwise specified, or to particular materials unless otherwise specified, and may incorporate only one or a plurality of the disclosed aspects of the disclosure and may further incorporate only one or a plurality of the disclosed features, and can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. As should be understood, any embodiment may incorporate only one or a plurality of the above-disclosed aspects of the display and may further incorporate only one or a plurality of the above-disclosed features. Moreover, many embodiments, such as adaptations, variations, modifications, and equivalent arrangements, will be implicitly disclosed by the embodiments described herein and fall within the scope of the present disclosure.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

All publications mentioned herein are incorporated herein by reference to disclose and describe the methods and/or materials in connection with which the publications are cited.

### A. Definitions

It is also to be understood that the terminology used herein is for the purpose of describing particular aspects only and is not intended to be limiting. As used in the specification and in the claims, the term “comprising” can include the aspects “consisting of” and “consisting essentially of” Unless defined otherwise, all technical and scientific terms used herein have the same meaning as commonly understood by one of ordinary skill in the art to which this invention belongs. In this specification and in the claims, which follow, reference will be made to a number of terms which shall be defined herein.

As used in the specification and the appended claims, the singular forms “a,” “an” and “the” include plural referents unless the context clearly dictates otherwise. Thus, for example, reference to “an assembly” includes two or more assemblies.

Ranges can be expressed herein as from one particular value, and/or to another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

The terms “first,” “second,” “first part,” “second part,” and the like, where used herein, do not denote any order, quantity, or importance, and are used to distinguish one element from another, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally affixed to the surface” means that it can or cannot be fixed to a surface.

Disclosed are the components to be used to manufacture the disclosed devices and articles of the invention as well as the materials themselves to be used within the methods disclosed herein. These and other materials are disclosed herein, and it is understood that when combinations, subsets, interactions, groups, etc. of these materials are disclosed that while specific reference of each various individual and collective combinations and permutation of these materials cannot be explicitly disclosed, each is specifically contemplated and described herein. For example, if a particular material is disclosed and discussed and a number of modifications that can be made to the materials are discussed, specifically contemplated is each and every combination and permutation of the material and the modifications that are possible unless specifically indicated to the contrary. Thus, if a class of materials A, B, and C are disclosed as well as a class of materials D, E, and F and an example of a combination material, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the articles and devices of the invention. Thus, if there are a variety of additional steps that can be performed it is understood that each of these additional steps can be performed with any specific aspect or combination of aspects of the methods of the invention.

It is understood that the devices and systems disclosed herein have certain functions. Disclosed herein are certain structural requirements for performing the disclosed functions, and it is understood that there are a variety of structures that can perform the same function that are related to the disclosed structures, and that these structures will typically achieve the same result. Further, the following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header. The present disclosure includes many aspects and features. Moreover, although many portions of the present disclosure are written with the reference to dental embodiments, it should be understood that the intraoral visualization system of the present disclosure may be adapted and applied in many different environments, and many other compatible uses.

### B. Intraoral Visualization Devices and Systems

As briefly described above, the present disclosure provides, in various aspects, an intraoral visualization device and system with integrated peripherals and/or imaging devices. This overview is provided to introduce a selection of concepts in a simplified form that are further described below. This overview is not intended to identify key features or essential features of the claimed subject matter. Nor is this overview intended to be used to limit the claimed subject matter's scope. The intraoral visualization system with integrated peripherals may be used by dentists or dental practice operators for intraoral visualization, image capture and transmission. In one aspect, provides herein is an intraoral visualization system including at least one intraoral visualization device, each intraoral visualization device comprising: a casing comprising a base and a head; at least one imaging device contained within the head, the at least one imaging device configured to capture video data; a plurality of lighting units contained within the head, each lighting unit comprising a discrete lighting source; a communications module; a power module; and at least one switch in operative communication with and configured to control operation of at least one of: the at least one imaging device or the plurality of lighting units.

In further aspects, the system can be integrated with a tele-dentistry platform configured to remotely treat a patient, for example, the KARE™ mobile dentistry platform. The multimedia visualization content captured and transmitted by the system may include video, audio, location and telemetry data. The system may be further configured to generate and/or determine other information, including, for example, diagnostic data. The content capture and transmission may be configured to occur, in certain embodiments, from a remote location, such as a patient's home. In turn, the system of the present disclosure would enable a tele-dentistry platform to remotely treat a patient using multimedia visualization content capturing and transmission.

Accordingly, embodiments of the present disclosure may provide an intraoral visualization system and device that can be securely mounted on, and be easily removed from, a patient. The system may be integrated with a plurality of sensing devices, including, but not limited to, cameras, telemetry sensors, lighting units, microphones and speakers (hereinafter referred to as “integrated peripherals”). The integrated peripherals may be grouped together and easily attachable and detachable to users. The cameras, imaging devices, and components may be configured to provide video capture. In addition, when used in conjunction with the speaker, the system may be used as a two-way communications system.

In various embodiments, the devices and system may be configured with additional electronics for telemetry data collection (e.g. temperature, biometric sensors, etc.) and patient diagnostic information. Moreover, the telemetry data collected may be streamed through the communications module as telemetry data along with its own metadata. Metadata may include, for example, but not limited to, device ID information, and be communicated in a separate stream. Moreover, in some embodiments, the telemetry data may be employed in the provision of 3-D renderings.

Devices and systems consistent with embodiments of the present disclosure may comprise electronic components including one or more of sensors, cameras, and lighting units, user interface components and wiring. The lighting units may include one or more lighting sources. In further aspects, the lighting sources can comprise one or more of UV, LED, laser, infrared, ultrasonic, and other radiation sources. In an exemplary aspect, the components can comprise an antenna (e.g., Built in IPEX antenna, Operating Frequency 2.4 GHZ, Data rate 802.11 b/g/n: up to 300 Mbps), image sensor (e.g., CMOS, CCD, etc.), lighting units (e.g., adjustable high intensity LED's (5600K, 6500K, etc.) and e.g., purple UV 80-150 mcd 395 nM black lights).

Further, in some embodiments, the disclosed devices and systems may be able to function in one or more of multiple available modes and sub-modes, including calibration mode(s), inspection mode(s), diagnosis mode(s), and treatment mode(s). For example, a calibration mode may include testing illumination, vision, or other sensors, capturing image or video data, visual projections, calibration on a treatment surface, or making calculations for alignment. For example, the UV lights can illuminate the teeth in a hue that will aid in identifying hairline fractures and other potential oral pathology that may be more visible with a contrasting illumination source.

In some embodiments, the system may employ an augmented reality (AR) system to assist an operator (e.g., dentist or dental operator) with operation of the system. An augmented reality system may, for example, project assistive features onto the treatment surface. In various embodiments, an augmented reality system can be calibrated by projecting one or more markers or patterns onto the treatment surface in addition or alternative to other calibration processes.

In some embodiments, the system may comprise a 3-dimensional vision system. Such a system might involve, but is not limited to, for example: stereoscopic cameras, one or more sensors (electromagnetic, acoustic, ultrasonic, etc.) that are depth-sensitive or otherwise capable of mapping spatial morphology of the mouth, or visual processing of 3-D features via controlled motion of items in the workspace. In various embodiments, a 3-D vision system can aid in performing procedures on surfaces having complex, non-flat morphology—such as teeth or the gum-line. In various embodiments consistent with system comprising 3-D vision capabilities, stereo vision may be utilized to automatically measure teeth or mouth dimensions. In further aspects, a controller, including a processing unit, can connect to an imaging device or camera (which can include illumination from lighting unit, e.g. an LED source), a user interface device (such as touch panel display), a power module, a control box, and auxiliary or service items (such as Wi-Fi/Bluetooth communication modules, other dental devices).

By way of non-limiting example, the tele-dentistry platform may be hosted on a centralized server, such as, for example, a cloud computing service. Alternatively, in some embodiments, platform may be implemented on one or more of the computing units. A user may access a tele-dentistry platform through a software application. The software application may be embodied as, for example (but not be limited to), a website, a web application, a desktop application, and a mobile application compatible with a computing device. Further, a user could control one or more operations related to performing dental procedure while operating the system.

Accordingly, platform may be configured to communicate with computing unit of the system over communication network. For instance, the platform may be configured to receive control inputs from user (e.g., dentist) in order to initiate an intraoral examination session. Accordingly, in some embodiments, the platform may communicate with a software application installed on the computing unit of the system. For example, in some instances, the platform may send command signals to the software application in order to control operations such as performing image captures, perform inspections, and perform diagnostics test.

Further, in some instances platform may also be configured to transmit configuration settings to be adopted during operation of the system. Accordingly, the computing unit of system may be configured to capture images along a path based on the received configuration settings. Any of platform(s), device(s), machine(s), and computing device(s) may be involved in the generation, transmission, storage, processing, enactment, or supervision of instructions and other operations. A computing device and/or the mobile device through which the platform may be accessed may comprise, but not be limited to, for example, a desktop computer, laptop, a tablet, or mobile telecommunications device.

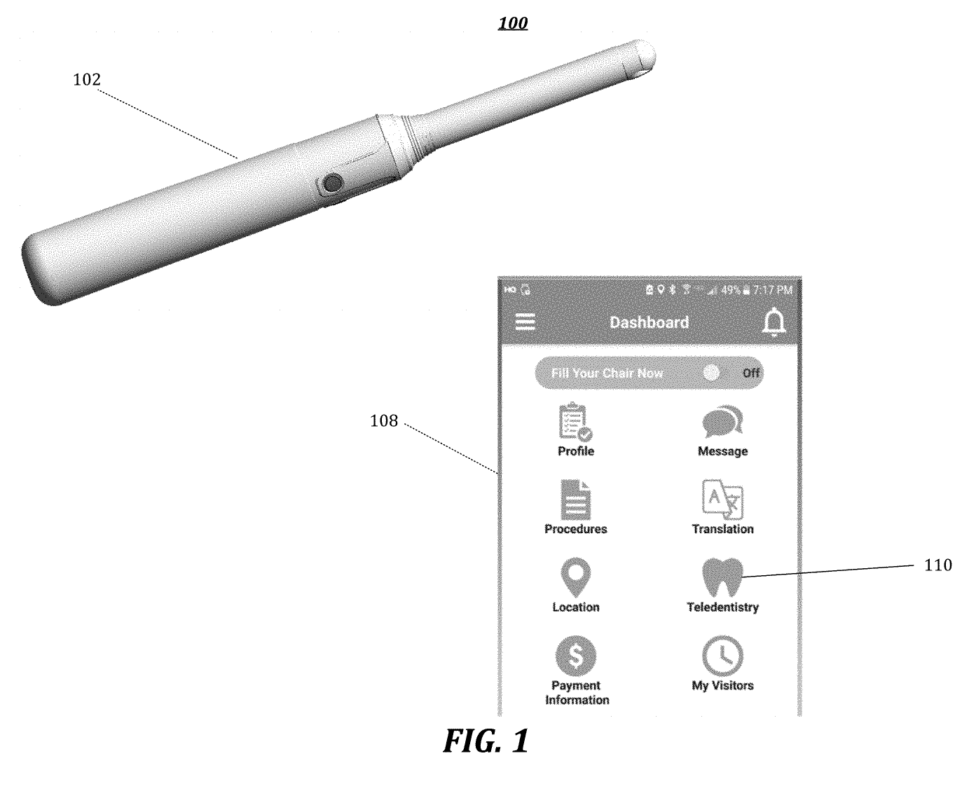

According to various aspects of the invention, intraoral visualization devices and systems of the present disclosure can comprise multiple configurations. In further aspects, FIG. 1 shows an exemplary embodiment of a system 100 for intraoral visualization.

As shown in FIG. 1, the system 100 comprises intraoral visualization device 102 and a control unit 108 in the form of a wireless device with an app and in operative communication with and configured to control operation of intraoral visualization device 102. The app on wireless control unit 108 provides graphical user interface 110 for using device 102 in connection with a tele-dentistry functionality and other functions of the platform, as further described herein.

In further aspects, the disclosed intraoral visualization devices and systems can comprise a plurality of integrated peripherals. In some embodiments of the present disclosure may provide systems that comprise a plurality of peripherals that are configured to be integrated into an intraoral camera or device. Yet further embodiments of the present disclosure may provide methods for integrating a plurality of peripherals into an intraoral camera or device.

In some aspects, the intraoral visualization system (interchangeably referred to as apparatus or device throughout this disclosure) may be configured to be positioned over or around a patient's mouth. In further embodiments, the system may be placed or held inside a patient's mouth.

In other aspects, portions of the system may have a unique identifier so that it may be associated with the patient or dental provider that operates the system which can, in some embodiments, be utilized in connection with payment and reimbursement in connection with dental services rendered on a patient. The dentist-system-patient association may occur on a remote, server end and be controlled and modified at any time. In this way, operators (e.g. dentist) can interchange systems, for example, to treat a different patient in a different location and/or network.

In further aspects, integrated peripherals consistent with embodiments of the present disclosure may comprise a plurality of devices configured to capture, for example, imaging or video content, light sensing devices, temperature sensing devices, and location data (hereinafter referred to as the “capturing devices”). In some embodiments, the capturing devices may be imaging devices, such as cameras. In other aspects, the cameras may be multi-depth focus cameras configured to detect multiple light fields. One such example of a camera may be a multi-array camera, or the like.

The integrated peripherals may further comprise a communications module comprising, for example, Wi-Fi and/or Bluetooth devices configured to stream the data. The data may be streamed in accordance to systems and methods disclosed in tele-dentistry platform applications, such as those disclosed herein.

Consistent with embodiments of the present disclosure, the invention is described as including one or more imaging devices. If the invention is used in system having a pair of intraoral visualization devices, generally, each of the intraoral visualization devices will have the same number of imaging devices, but not necessarily so. In still further aspects, other embodiments of the invention, in contrast, may provide one of the intraoral visualization devices with more imaging devices than the other. In yet further aspects, the one or more imaging devices in each intraoral visualization device need not be identical. For example, according to an embodiment, one of the intraoral visualization devices has stronger or more robust imaging devices than the other, differently sized imaging devices than the other, differently positioned imaging devices than the other, etc.

In various aspects, intraoral visualization devices may further comprise a housing or casing configured to house one or more components. In further aspects, the casing may be configured to contains at least one imaging device and at least one lighting unit. In some aspects, a casing or at least one surface of a casing, may be shaped or curved for easy gripping. The casing may be manufactured of a rigid material such as for illustrative purposes a plastic, metal or wooden casing, wherein the casing is a container with walls to define an enclosed area. Other flexible or pliant or other materials may be employed.

In further aspects, the casing can be any shape, and may be in the shape of a three-dimensional polygon and the casing walls may define an interior space or interior sections for containing the operating elements of the invention. Any other shape (as used herein, the term shape is used in the broad sense of three-dimensional works) may be employed, so long as the shape is large enough and structured so as to be able to contain the various working components of the invention and use within intraoral cavity as more fully disclosed below.

In further aspects, the imaging device may cooperate with one or more lighting units to capture and visualize the subject. The placement of the imaging device and/or lighting units can be variable so long as the illuminating effects of the lighting units can produce the desired image data. The placement of the imaging device within the casing is variable so long as the imaging device can capture the desired portion of the mouth. In still further aspects, the walls of the casing define an interior space that, is sized to contain at least one imaging device, at least one lighting unit, wiring to connect components and a power module.

In further aspects, the imaging devices and/or lighting components can be connected to or otherwise disposed on an outer surface of the casing. In still further aspects, the imaging devices and/or lighting components can be integrally connected between an outer surface and inner surface of the upper. In various aspects, the imaging device arrangement and construction can be configured to correspond to areas of a user's mouth where treatments are greatest.

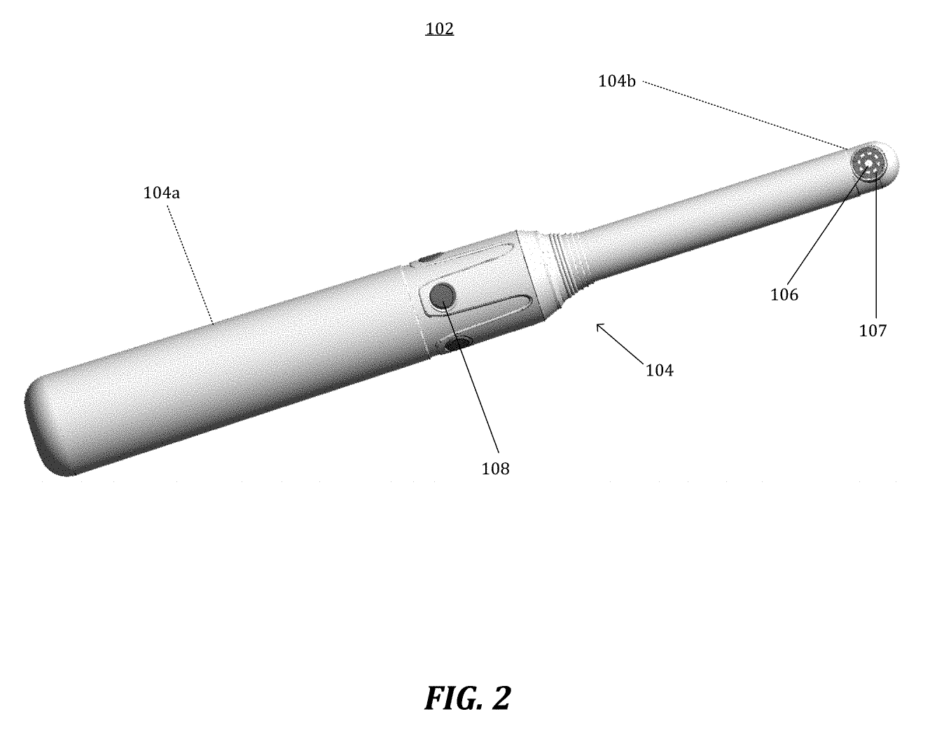

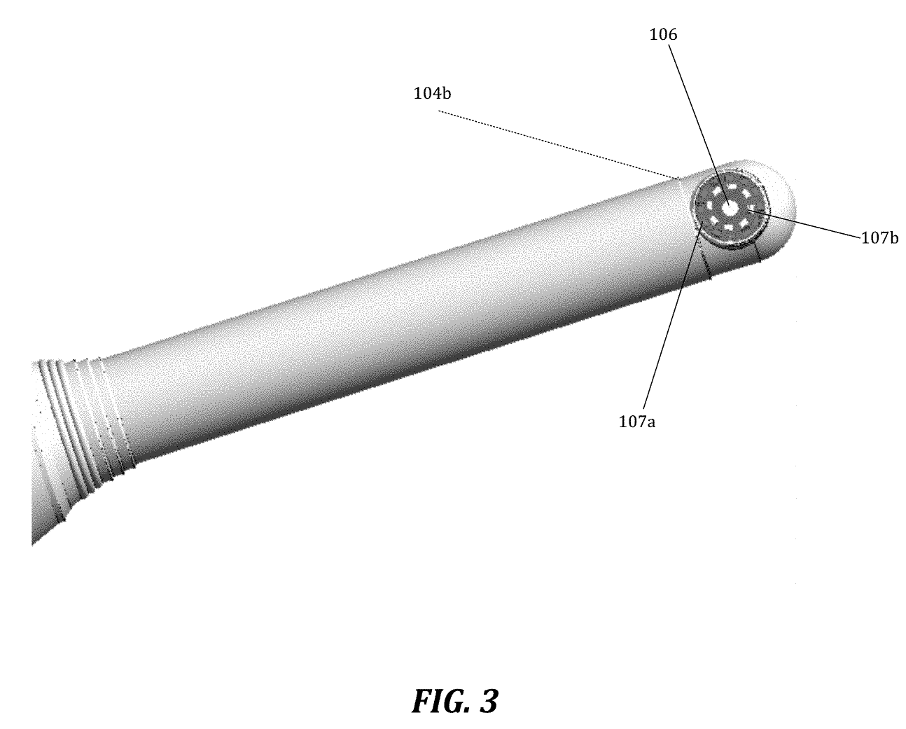

FIGS. 2 and 3 show an exemplary embodiment of an intraoral visualization device 102 in accordance with the present invention. As shown in FIG. 2, the intraoral visualization device 102 comprises a casing 104 comprising a base portion 104a and a head portion 104b; an imaging device 106 contained within the head, a plurality of lighting units 107 also contained within the head, and control units 108 in the form of a plurality of switches in operative communication with and configured to control operation of device 102, including but not limited to operation of imaging device 106 and lighting units 108. As shown in FIG. 3, intraoral visualization device 102 can comprise two sets of lighting units 108a, 108b contained within the head 104b, each lighting unit comprising a discrete lighting source, for example, one lighting source being white light and the other being black light.

In further aspects, one or more lighting units can be included in each intraoral visualization device. In still further aspects, each intraoral visualization device may comprise a plurality of lighting units. In still further aspects, illumination effects can be provided by any desired lighting source such as, for illustrative purposes, a lighting unit provided within the casing. In yet further aspects, the lighting unit may comprise one or more lighting elements. In still further aspects, the lighting unit may comprise a plurality of lighting elements. In even further aspects, all or a group of the plurality of lighting elements can correspond to an illumination bank. Once illumination is activated by providing power to the lighting units and elements, the illumination may be constant and continual, or the illumination may be intermittent, and cycle on and off at the same or a different illumination speed or frequency.

In further aspects, the lighting units and/or elements can be of any desired lighting source or means for producing illumination. Non-limiting examples of suitable lighting elements include LEDs, and the like. It is only important that the lighting element be able to produce the appropriate lighting type and illumination to the subject at a sufficient level to produce the effect intended in the disclosed visualization, diagnosis, and treatment methods.

According to various aspects, the disclosed intraoral visualization devices and systems comprise, but not be limited to, at least one controller and/or on-board computing module. In further aspects, the controller may be coupled to the capturing devices and the communications module. In this way, the controller may communicate, bi-directionally, data captured by the system as well as operational data received from a remote location. The controller may comprise a micro-embedded processor placed within the system. For example, the controller may comprise a system on a chip (SOC), and other like chipsets. A quantity of processors (or processing modules) that may be integrated into the system may depend on a quantity of capturing devices.

In still further aspects, controller and/or computing module may be in operative configuration and communication with, for example, but not be limited to, imaging sensors, lighting units, activating switch, communication module, power module, various telemetry sensors, transceivers and antennas. Further, the computing module may be in operative communication with another computing device consistent with the description herein, and may comprise, but not be limited to, a wireless device, smart phone, desktop computer, laptop, a tablet, or mobile telecommunications device. Such remote devices may be used to control and/or configure integrated computing module (e.g., activation conditions, image capture operating parameters and settings, and the like). Moreover, the disclosed intraoral visualization devices and systems may be in operative communication with a centralized server, such as, for example, a cloud computing service. In still further aspects, controller may comprise a PCB board for the System-on-a-Chip (SOC) and related electronics, or an additional processing module. The additional processing module may comprise an additional PCB board for an additional SOC related electronics. The system may share the input-output to all the integrated peripherals (e.g., cameras, speakers and microphones). In some embodiments, the controller may be configured to operate a first set of capturing devices (e.g., front-facing cameras), while the additional processing module may be configured to operate a second set of capturing devices (e.g. side-facing, rear-facing, and/or second front-facing cameras). The system may be designed with different configurations based on different peripheral device integration layouts. In various embodiments, the controller may further house a plurality of sensors, such as, for example, but not limited to, GPS location, accelerometers, RFID, and the like. In some embodiments, a proximity sensing device, such as, for example, the RFID may be used to operate the capturing devices in a power-efficient manner.

In yet further embodiments, the location of the device (e.g., grouped peripherals, and as may be detected by a location detection module) may be correlated with its operation (e.g., confirm correct patient and/or location) based on information and/or data a centralized server is attempting to provide or confirm. The Wi-Fi antenna, which may be a part of the communications module, may be used to stream the content over Wi-Fi, although Bluetooth communication devices and protocols may be used as well. The shape of this antenna may be similar to that found in smart cell phones that require good Wi-Fi connections, such as, for example, the Apple iPhone.

In some embodiments, it may be preferred to place cameras in position where all the angles from each image capture sensor overlap to provide a hemispheric view. In other embodiments, the content captured by the cameras may be stitched together, a panoramic view may be provided around a vertical axis, relative to the ground, to represent the left to right motion. The batteries that may be used to power the integrated peripherals of the intraoral visualization system should be of sufficient capacity to last at least the duration of a dental treatment session in which the system is employed. The capacity of batteries may be, for example, smaller when used in conjunction with RFID or other remote power management techniques that place the electronics in standby/lower-power mode and preserve battery life.

The intraoral visualization device and system may further comprise a power module. The power module may include, for example, a battery. The battery may be replaceable and/or rechargeable. The embodiments where the battery is chargeable, the power module may include a port for receiving a power supply. In various embodiments, the battery may be integrated into the intraoral visualization device itself. In other embodiments, where longer battery life is required, the battery may be external to the system parts configured for intraoral use in order to, for example, accommodate larger battery size.

In some embodiments, a Wi-Fi antenna may be used to stream data, including, for example, but not limited to, the video, telemetry, diagnostic, and sensor data, over Wi-Fi. The shape of the antenna may be similar to that found in smart cell phones that require good Wi-Fi connections, such as the Apple iPhone. In some embodiments, a Bluetooth connection may be used instead of, or in conjunction with, Wi-Fi communication. The data may be streamed to a centralized server where the data may be processed. A computing device, which in some embodiments, may be contained within the controller to pre-process the captured data.

In further aspects, the disclosed devices and systems can work in one of multiple available modes: 1) calibration, 2) inspection, 3) diagnosis, and 4) treatment. During calibration mode, the system can ensure that the peripherals, sensors and/or components are properly calibrated and/or aligned or ready for operation. The stages involved in calibration may include: 1) illumination/sensor testing, 2) calibration on a test surface, and 3) making calculations for alignment.

Although the present methods of using the disclosed devices and systems have been described to be performed by computing device, it should be understood that, in some embodiments, different operations may be performed by different networked elements in operative communication with computing device. Various stages in the present methods may be performed by at least one of an intraoral visualization device, its vision system stage, and its operator. An operator may perform stages using various interface elements available on the intraoral visualization device or system. The lighting units can be synced with the frequency of ambient lighting (which may be subject to oscillations related to, e.g., the 50 Hz or 60 Hz frequency of the power grid). Next, the system can acquire an image of the mouth via imaging sensor of the imaging device or camera. The image parameters can then be analyzed at to determine whether the acquired image is acceptable (whether illumination is sufficiently uniform, proper white balance, etc.). If the image is determined to have an issue—for example, insufficiently uniformity or too dark—adjustments can be made to illumination banks (to improve uniformity) and to exposure and gain (to improve brightness and white balance). Other adjustments may be made as well. In various embodiments, adjustments are made until certain threshold parameters are met for one or more of exposure, gain, and intensity. Thereafter, based on the adjustments made, the method can compute optimal values for exposure, gain, and intensity. The calculated values are received, and reported back for next command. The system can learn and retain non-uniform illumination information—i.e. that which can't be corrected by adjustment, or others (if any).

In some embodiments, calibration stages (calibrate illumination, calibrate vision, and potentially other calibration stages like calibrate AR) may be performed in one phase, in a different order, or with additional or fewer stages. The method may include initializing image capture and acquiring an image of the mouth to verify Illumination to make sure no variables were changed since earlier calibration. Based on the image and coordinates of identified elements, the method can include performing one or both of calibrating for distortion caused by the optics, and determining the location of the camera relative to the origin of the workspace (e.g. mouth) to complete the vision calibration. In various embodiments, the method can be employed in the context of an augmented reality or projection system. Such a system may be used to project assistive features into the workspace, such as patterns, guides, system status information, or virtual controls.

Many embodiments can utilize one or more calibration stages that seek to achieve optimal workspace qualities for the system. In addition to, in combination with, instead of, before, during, or after such calibration stages, the intraoral visualization device may perform a calibration of an augmented reality system. In various embodiments, such a calibration can be consistent with the present methods. In further aspects, the method may be utilized in embodiments consistent with an intraoral visualization device having a 3-D/depth-sensing/stereoscopic vision system. For example, spatial information about the topology of items in the mouth may inform the system about the proper calibration of augmented reality features, projection coordinates, lighting parameters, etc.

Embodiments consistent with an intraoral visualization device comprising an AR system may be of particular benefit when examining or treating patients, as the assistive features may improve the quality and consistency of the dental treatment. Such AR systems may also lower costs due alleviating the need for certain special examination tools, as well as due to error. In embodiments that comprise the ability to visually present, within the workings are of the device, textual or other status information/indicators to the dentist operator, such “heads up” style presentation may be beneficial to operators performing an oral examination. One reason is that the operator may not have to look away to view (and in some embodiments, interact with) device controls. In an example, an AR system might project a certain symbol, color, or textual warning to alert an operator to “examine an area” or “potential pathology” during operation. In another example, after performing a procedure or diagnostic action, an AR system may project information about the results of the action and may highlight any issues found. Embodiments comprising AR features may also interact with other features of an intraoral visualization device. For example, AR may involve or interact with a gesture recognition feature. This may be beneficial, for example, when using the AR projections as a “virtual interface” with which the operator might interact on or about the surface of the patient treatment area.

Augmented reality systems may, in some embodiments, be achievable with—or integral within—an extant visual system (optics, lighting, sensors, etc.) In other embodiments, AR features may necessitate one or more additional, dedicated, or discrete components. In further aspects, projections may be monochromatic or colored, and may include features that are straight, curved, irregular, text-based, temporal (e.g. blinking), etc. Projections in various embodiments may be made with visible light, infrared light, other electromagnetic radiation. Embodiments may comprise augmented reality features that are visible to the naked eye. Some embodiments may comprise AR features that are visible through an AR viewport such as a smartphone configured for VR/AR use. A method can comprise projecting assistive features onto a patient treatment area in order to guide the procedure in the proper location and orientation.

In various embodiments, the method may include the addition of one or more stages of detecting oral pathologies, or other objectionable dental imperfections. If treatable, such area can be treated. If not, an operator may capture an image to help with determining treatment options. In further aspects, the method can project assistive features onto a workspace (e.g., teeth or mucosa, etc.) in order to guide the placement of a crown, bridge, implant, or the like in the proper location and orientation. While AR assistive features can be beneficial with the placement of a relatively simple prosthetics, but may be of even greater benefit when placing multiple and or/complex prosthetics. The intraoral visualization device and system may thus provide visual guidance to an operator (or, as is generally possible in various embodiments, to another device or automated system) as to correct order, location, and orientation of dental prosthetic installations or treatment. In still further aspects, a method, consistent with embodiments having depth-aware or 3-dimensional vision systems, may use various methods to achieve z-axis (height off the workspace) spatial awareness of the working area, such as dual (stereoscopic) or multiple cameras, electromagnetic sensors, processing of controlled motion image captures, and the like. In the context of an embodiment consistent with an intraoral visualization device, the method may comprise a verify stage configured to detect deviations in a placed and imaged treatment from its known-correct exemplar. Upon imaging the placed treatment, method can, upon detecting issues, alert the operator that the treatment quality does not verify.

In further aspects, the invention may include embodiments having different features with respect to the placement and/or positioning of the imaging devices in the intraoral visualization devices. In some embodiments, a user may be able to choose where to position the imaging device with respect to a patient's tooth. In other embodiments, the system may automatically position the imaging device and the user may have limited ability to position and/or adjust the imaging device. In further aspects, the imaging device may be fixed or integrated with respect to the casing so the user may have little, if any, choice in positioning of the imaging device, and thus would rely on repositioning the intraoral visualization device within the mouth cavity.

In some embodiments, the invention may provide an app on a wireless device configured to communicate with each of the intraoral visualization devices. In further aspects, each of the intraoral visualization devices may require the appropriate elements to receive, act on the communications from the wireless device, and/or respond to the app with information. As described herein, in various embodiments, the invention may include a controller to receive the communications from a wireless device, to pass them on as instructions to the intraoral visualization devices, to receive data and/or information from the intraoral visualization devices, and/or to send the data and/or information to the app on the wireless device. The controller may include elements to carry out its function. For example, the controller may include a receiver/transmitter, transceiver, and/or antenna for communicating with the wireless device. The controller may include processing technology such as a microprocessor, etc. to process and execute the instructions, data and/or information, and/or signal received from a control unit or wireless unit or the data and/or information received from the intraoral visualization device. As another example, the controller may be connected respectively by one or more wires (and/or other transmitters or carriers) to the one or more intraoral visualization devices of system to transmit instructions/data/information to the intraoral visualization device and/or to receive data and/or information from them.

In further aspects, while the imaging devices and/or lighting units described herein can be permanently mounted in or on intraoral visualization device or device structure, this is not a requirement. For example, the imaging devices and/or lighting units can be connected to or disposed on a detachable device head configured to be removably coupled to a device base or casing base, e.g., to allow interchange and/or replacement of the imaging devices and/or lighting units. To this end, one or more imaging devices and/or lighting units can be detachably connected to the device base, e.g., to allow interchange and/or replacement of one or more imaging devices and/or lighting units (individually or as a unit with device head). Such configurations allow users, dentists, retailers, or others to select desired imaging and illumination properties or levels, e.g., for customization purposes, for treatment indications, to match desired treatment use, a patient's physical characteristics, a patient's symptoms, or to repair or replace defective or damaged imaging devices and/or lighting units, etc.

In further aspects, the disclosed intraoral visualization devices and systems may further comprise one or more of the following components: a voltage regulator, power switch (e.g., MOSFET), power management module, battery management module (e.g., fuel gauge), battery charging module, wireless power coil or receiver, wireless power control module, antenna (e.g., Bluetooth LE antenna), transceiver (e.g., Bluetooth LE transceiver), interface module, control module, voltage sensor, current sensor, power input, and magnetic switch.

In some embodiments, the intraoral visualization device and system can have a wireless charging system comprising a battery charging module, wireless power receiver (e.g., wireless power coil), and wireless power control module. In some embodiments, the controller and above component can be located in the same area of the device casing, for example, disposed within the base. In further aspects, the user can simply set the base on a compatible wireless charging mat or cradle to charge the battery.

In various aspects, the components of the disclosed devices and system can be detachably attached. In further aspects, the components can be connected by a connecting means. In still further aspects, the connecting means can comprise a fitting, insert, adhesive, brazing, soldering, welding, spot weld, screw with nut, rivet, threading, friction fit, snap-fit, twist-lock, or interlocking mechanism or a combination thereof. In yet further aspects, the connection can be achieved using a snap, friction fitting, snap ring, O-ring, pressure fitting, clip, clasp, and the like. The snap ring or O-ring can be retained within a groove to accommodate the snap ring or O-ring. In a further aspect, the system can comprise an engagement means for coupling and holding components together. In a further aspect, the engagement means can be a screwing mechanism, a click-lock mechanism, or friction mechanism, or the like.

In further aspects, the device and system components can be integrally or mechanically attached to other components. In a yet further aspect, the disclosed components can be connected, attached, or mounted using a connecting means, the connecting means comprising a fitting, insert, adhesive, brazing, soldering, welding, spot weld, screw with nut, rivet, fitting, insert, threading, friction fit, or snap-fit or a combination thereof.

In further aspects, application software (an app) on a wireless device such as a mobile phone may be used to activate and de-activate and/or otherwise control the operation of the intraoral visualization device. In still further aspects, the app may comprise a tele-dentistry platform and/or tele-dentistry functionality. In yet further aspects, the disclosed intraoral visualization devices may be equipped with wireless technology to communicate with the app on the wireless device. In even further aspects, the app may provide at least ones of: a graphic image of the user's mouth and/or teeth to indicate where (and/or other characteristics of the mouth) the device(s) are visualizing and/or diagnosis the patient's mouth at the time of operation, possible areas of the patient's mouth to indicate where (and/or other diagnostic characteristics) treatment may be needed, telemetry data received from the sensors in the devices.

Also disclosed herein is a mobile and/or tele-dentistry platform which, in some aspects, can be used to provide remote dental services. In further aspects, the platform may be used by users (e.g., patients) to match with providers (e.g., dental providers) based on predetermined criteria in order to receive dental care services (e.g. dental exams, procedures, cleanings, or the like).

In still further aspects, the platform may enable users and providers to create a profile. The user profile may include user information or data such as, for example, information related to location, name, address, email, age, gender, phone, insurance information, payment information, medical history, dental history, DOB, symptoms, frequency, requested procedure, symptoms, time, calendar data, photographs, images, videos, and/or audio files. The provider profile may include provider information or data such as, for example, information related to office locations, name, address, email, age, gender, phone, NPI number, Medicaid provider information, third party insurance provider information, medical and/or dental credentials, license information, experience history, dental practice type and specialties, procedures and services offered, time, calendar data, photographs, images, videos, and/or audio files. In yet further aspects, the platform may then match the patient with providers based on both the users' profile data and/or other requirements, as further described herein. In even further aspects, the platform may enable its users to search for known providers matching their requirements and/or based on the users' proximity to each other. In still further aspects, after establishing a patient, each platform user may review the user's profile and interact via the platform, such as by sending a message.

In further aspects, the platform may process profile data based on matching rules which may include at least one of: analyzing predetermined selection criteria provided by a first user with profile data from the plurality of providers; analyzing a first user's profile data with profile data from the plurality of users; and analyzing predetermined criteria provided by the plurality of providers with the first user's profile data. In still further aspects, processing the profile data based on matching rules may include at least one of: analyzing predetermined criteria and/or profile data provided by a first user with profile data from the plurality of providers to identify a set of matching providers; analyzing predetermined criteria provided by the plurality of providers with the first user's profile data to identify a set of matching providers; reducing a size of a matching provider set by applying at least one filter; and reducing a size of a matching provider set by applying at least one filter. In yet further aspects, the least one filter may include at least one of: applying a matching algorithm; applying a folding routine; eliminating matching users from a matching user set having profile data comprising a predetermined parameter; eliminating matching user groups from a matching user group set having profile data comprising a predetermined parameter; and eliminating matching providers that are present in a controlled library; and eliminating matching user groups that are present in a controlled library.

In further aspects, the platform may determine profile data may comprise at least one of: location, group, organization, club, school, name, age, gender, at least one activity of interest, activity skill level, activity frequency, time, event, calendar data, at least one photograph, image, video, and audio file. In still further aspects, determining the profile data can comprise determining, by the platform, profile data based on a user's answers to a questionnaire. In some aspects, determining the profile data can comprise determining profile data based on a first user's answers to a questionnaire. In yet further aspects, determining the profile data can comprise receiving profile data from a third-party platform comprising information associated with a user. In even further aspects, determining the profile data can comprise receiving profile data from a third-party platform comprising information associated with a user. In some aspects, profile data of the plurality of providers may be determined using a user's answers to a questionnaire. In other aspects, profile data of the plurality of providers may be determined using data from a third-party platform comprising information associated with the plurality of providers.

In further aspects, the platform may notify one or more users. In still further aspects, notifying may comprise at least one of: notifying a user of at least one matching user and/or user group; displaying a set of matching providers and/or a set of matching user groups. In still further aspects, notifying may comprise at least one of: displaying a location of at least one user from the set of matching providers in a graphical interface, such as a map; displaying the location of a plurality of providers from the set of matching providers in a graphical interface, such as a map. In even further aspects, the graphical interface may be a dashboard configures to display various textual or visual representations of the platform and data. In some aspects, the displayed providers may be based on a first user's proximity to the displayed providers. In other aspects, the displayed providers may be represented on the graphical interface using a symbol, character, image, or the like. In various further aspects, selecting a displayed user may further comprise displaying the selected user's profile.

In further aspects, the matching or displayed providers may be organized or aggregated using at least one common or matching criteria selected from at least one of: location data, activity type, activity data, group data, group type, user group type, activity data, activity type, calendar data, duration, start time, stop time, and custom criteria provided by a user. For example, the matching or displayed providers can be aggregated together by location to see other relevant information of providers in the desired area, and then by activity type, and so forth. In still further aspects, a list of matching or displayed providers can be modified by applying one or more filters. In yet further aspects, the display filter can include at least one of: applying a matching algorithm; applying user selected options; hiding displayed providers having profile data comprising a predetermined parameter selected by a user; hiding displayed providers that are present in a controlled library; and hiding displayed user groups having profile data comprising a predetermined parameter selected by a user.

While the providers and procedures can be chosen from a list, users can create and/or request new services, which can then be grouped under preexisting procedure categories. In still further aspects, a user can transmit a connection request or invitation to a provider to establish a patient-provider relationship, such as to one or more unmatched providers. In even further aspects, patient-provider relationship provides definable platform privileges associated with user interaction, communication and platform activity. For example, if a first user updates their medical history in the platform, it will show the update, and any other platform user authorized to view the first user's platform activities, such as users belonging to provider group, for example employees of that particular dental practice.

In various aspects, the platform can enable user interaction between users (e.g., patients and providers) of the platform. In still further aspects, enabling interaction can comprise enabling a first user and the set of matching providers to interact with one another. In still further aspects, the platform can further comprise facilitating a communication between a first user and at least one provider from the set of matching providers. In yet further aspects, the platform can further comprise facilitating a communication between the first user and a plurality of providers from the set of matching providers. In some aspects, facilitating a communication may comprise enabling a first user and at least one user from the matching user set to schedule a shared activity or event. In other aspects, facilitating the communication can comprise enabling a first user and a plurality of providers from the matching user set to schedule one or more activities or events.

In further aspects, user interaction can comprise at least one of: transmitting a message, transmitting a post, sharing content, and the like. In still further aspects, the content can comprise at least one of: textual, graphical content, video content, and audio content. In even further aspects, enabling the first user can comprise providing the first user with a listing of activities. In still further aspects, providing the listing of activities can comprise providing at least one of the following: locations; group information, events, activities, and venues; or the like. In yet further aspects, the platform can further comprise presenting data and information related to the activities.

In some aspects, the profile data can comprise at least one of: location, group, organization, club, school, name, age, gender, at least one activity of interest, activity skill level, activity frequency, event, calendar data, and at least one photograph, image, video, and audio file. In other aspects, the predetermined criteria can comprise at least one of: location, group, organization, club, school, name, age, gender, at least one activity of interest, activity skill level, activity frequency, event, duration, start time, stop time, date, day(s), user, user group, photographs, images, video, audio; and the like.

In further aspects, the selected predetermined criteria or profile data can be changed at any desired time. In still further aspects, the system is configured to update matching providers and/or activities in real time in response to changes in the predetermined criteria and/or profile data and display updated results. For example, changes in location and/or activity type may cause the matching providers and/or activities to be updated. In yet further aspects, each predetermined criterion can have a predetermined time duration or time period.

In various aspects, the predetermined criteria can have at least one predetermined time duration or time period. In further aspects, the predetermined time duration can be from about 1 minute to about 24 hours, or more. In still further aspects, the time period can be a period between 12 A.M. and 12 P.M., for example, from 4 P.M. to 6 P.M. In yet further aspects, a plurality of predetermined time durations or time periods can be selected. To this end, each predetermined criterion can have a predetermined time duration or time period. For example, a user can select a limited time period for a given location which corresponds to the time period that the user expects to be in that given location and can select a different time period or duration for certain procedure type, such as a longer procedure, such as root canal. In further aspects, the time parameter of the platform can enable a user to find appointment matches with providers the moment of searching, or the can select search activity parameters that include a time in the future and be matched with any provider who has the same time frame and other matching criteria. In some aspects, the platform can utilize user calendars for events or future events. To this end, the platform can take advantage of matching a patient and provider's calendar linked to the platform.

In various aspects, the platform is enabled for user interaction between one user and at least one other user. In further aspects, enabling can be user interaction between the first user and at least one provider from among the set of matching providers. In yet further aspects, enabling can be user interaction between providers within a user group. In some aspects, user interaction can comprise at least one of: transmitting a message, transmitting a post, and sharing content. In other aspects, the content can comprise at least one of: textual, graphical content, video content, and audio content.

Platforms of the present disclosure may enable users to schedule dental services, for example, by using combinations of location, procedure, and provider data. In further aspects, upon selection of a feature of the platform that one user would like to schedule a procedure, the platform may provide the user with a listing of providers and/or procedures that match with that user. The listing of procedures may be an up-to-date listing of certain appointments that may be of interest to the users.

The platform may provide benefits including: time, users will spend less time searching for providers; convenience, as a user's schedule may require the procedure to be done at a time that providers are unavailable; a user is new to a city/locale—has just moved there or in-town on business—and doesn't know anyone to perform the procedure; and knowing who, what, and where at a glance.

Although the present disclosure references “procedures”, it should be understood that “procedures” can include examinations, consultations, appointments and expansions and modifications rendered thereto are within the anticipated scope of this disclosure. In various aspects, an appointment can comprise a procedure having a defined time period, duration, start time and/or end time. To the end, appointments may appear within the platform for fixed period of time, and no longer appear after the end time.

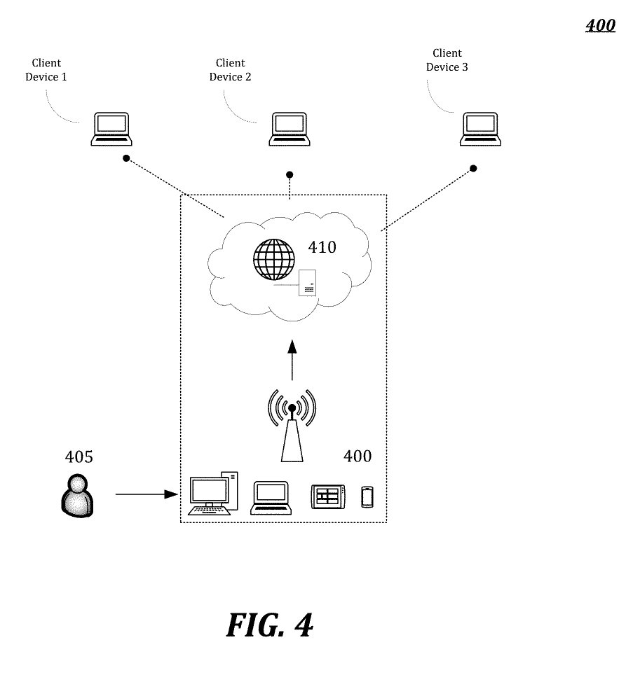

In various aspects, FIG. 4 illustrates one possible operating environment through which a tele-dentistry platform consistent with embodiments of the present disclosure may be provided. By way of non-limiting example, a platform 400 may be hosted on a centralized server 410, such as, for example, a cloud computing service. A user 405 may access platform 400 through a software application. The software application may be embodied as, for example, but not be limited to, a website, a web application, a desktop application, and a mobile application compatible with a computing device.

Platform 400 may provide an online interface for enabling users to interact with the platform. Such an online interface may be configured to receive user login and profile information. Further, the online interface may be configured to enable a user to search through his or her provider matches and interact with matched providers and other user within his or her corresponding user groups.

Moreover, platform 400 may provide an application interface that may be accessed with devices such as, for example, a desktop, laptop, smartphone or tablet device. The various applications that are adapted to each of these devices may be configured to communicate with server 410 perform the same function as the aforementioned online interface. In embodiments where the device has location tracking technology, the application may provide the platform with location information using location detection methods associated with the device, such as, for example, GPS, Wi-Fi and cell tower triangulation.

In addition to the platform interfacing with users through stand-alone website or application, or it may interface with users through social media as a social media app/plug-in/or software otherwise compatible with a social media Application Programming Interface (API). In this way, employing the API, the platform may retrieve user information (e.g., friends, interests, events, and the like) form a third-party social media platform (e.g., Facebook). Furthermore, the platform may interface with users through an administrator platform that may provide additional privileges to administrators.

In further aspects, a method performed by the platform may begin where platform may receive user information and/or input data from a user. For example, a user may create and register a profile. The profile may contain user information and data, and may receive at least a portion of this information by providing a questionnaire and receiving responses from the user. In some embodiments, the profile information may be populated from data received from a social media platform. For example, as mentioned above, the platform of the present disclosure may be configured to interface with a third-party social media platform over an API. Employing the API, the platform may retrieve user-data that is available to the social media platform. In this way, the platform user may select to have, for example, their Facebook LinkedIn, or Instagram profile data available for use with the present application.

In some embodiments, platform may receive information from one or more “collaborative calendars” and/or information entered by the user. For example, platform may receive information from Google Calendar. Such information may include scheduling data, such as information related to a time of an appointment and/or location for the appointment. The location may be, for example, an address, place or a set of GPS coordinates. Further, the platform may receive user current location information. For example, the platform may receive user GPS coordinates from the user's mobile phone. Such information may be used to predict when and where a user may for matching providers and tracking patient in route to appointments.

Platform may process data, such as, based on predetermined criteria, selection criteria or parameters, matching rules, and the like. For example, platform may match users with providers based on the extent to which the provider data from the user correspond with each provider. Matching rules may comprise excluding users from matches whose profiles do not match each other's preferences and/or excluding providers whose profile data does not match a user's provided profile data. In addition, matching rules may comprise a proprietary algorithm that corresponds to type and quantity of preferences that match between users.

Once platform processes data based on rules, a method may notify or display a set of matching providers. For example, the platform may present on one user's device a list of matching providers and/or a list or map of providers. The platform may first present on a first user's device a list of matching providers, and then present a list or map of matching providers and/or open appointments based on the first user's selection criteria. The list and/or map may be organized according to user preferences. Further, the platform may enable the user to select one or more of his matches to receive more information about the selected matches. In addition, the platform may notify a provider of a new match, for example, with a message, sound or vibration from his or her device which is connected to the platform 400.

In various aspects, a dashboard may be provided to display various visual representations of the match results. In further aspects, a standard set of views and templates may be used to display the matched providers and appointments and/or the associated data. In still further aspects, the dashboard may allow users to set configuration properties to customize the customized visual presentations, views, graphs, and/or charts. In yet further aspects, these configurations may include, for example, filter feature, an attribute identifier, single or multiple, aggregate function, default and customizable labels. The dashboard may also include the ability to define baselines for various parameters (e.g., location or time period), which may be used to provide real-time notifications relating to matching providers and/or procedures.

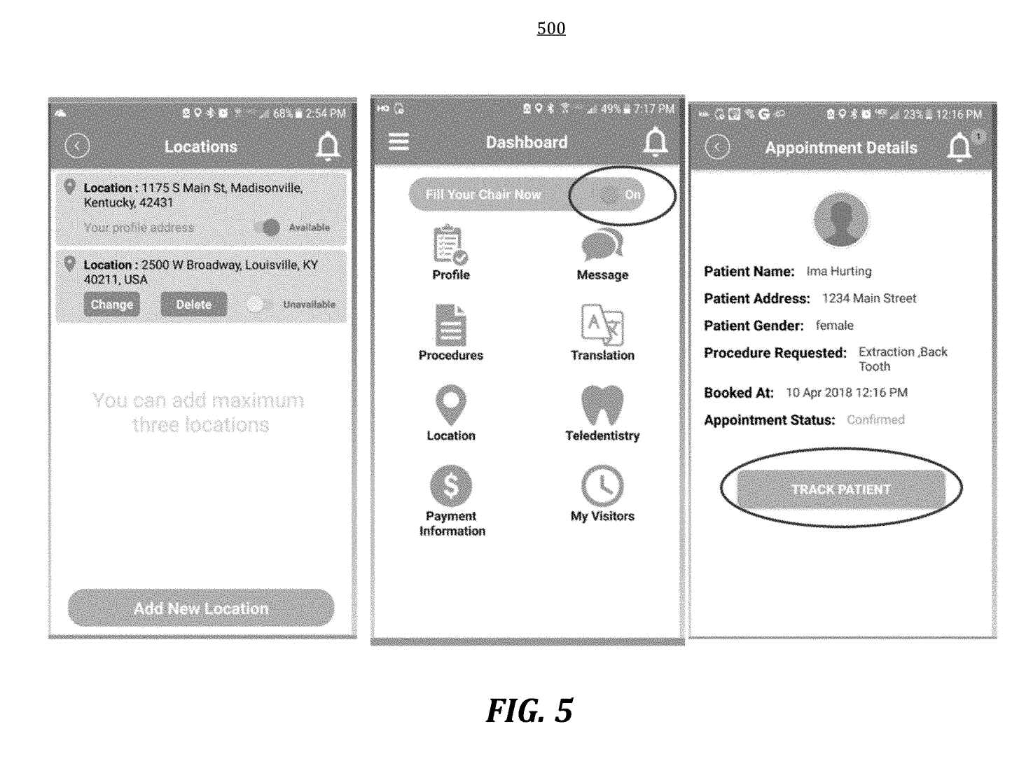

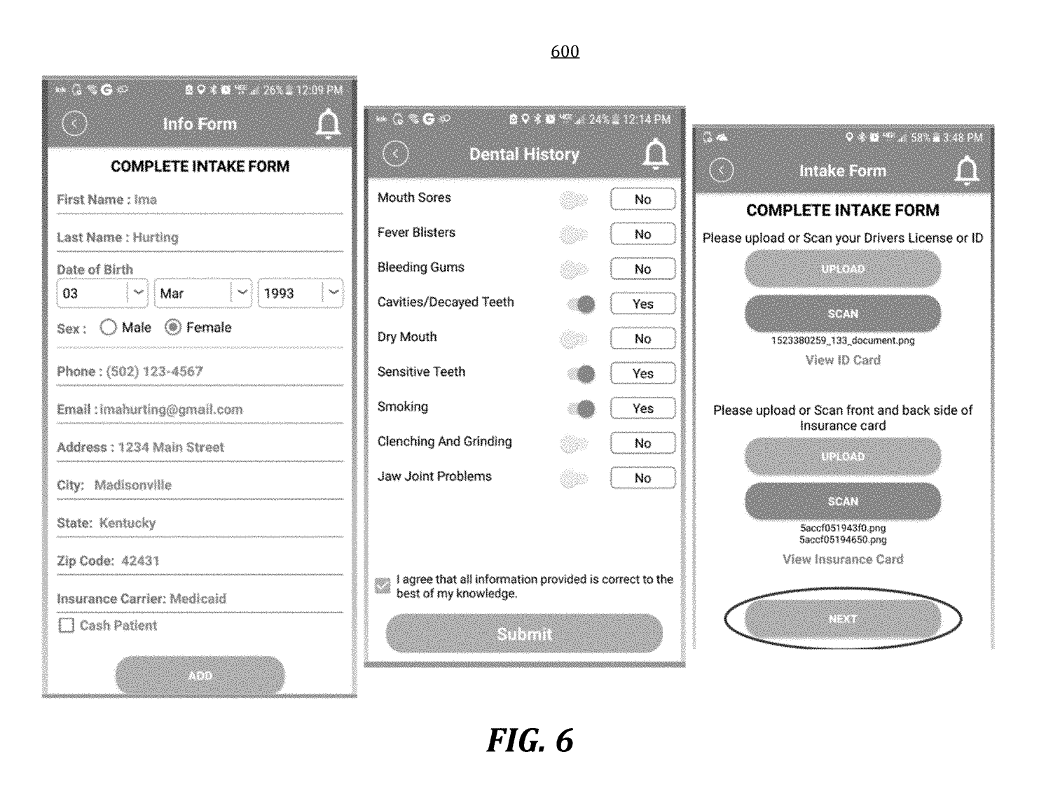

After platform notifies users of a match, platform provide a platform for interaction between users. For example, the platform may provide a direct messenger between users. Further, the platform may present the users with information pertaining to their preferred procedure, such as, for example, location, rules, advertising, special offers, pricing, promotions, tips, articles, pictures and other information related to the procedure. In some aspects, upon providing the platform with an indication that one patient would like to book an appointment with a provider, the platform may provide the users with notification upon confirmation by the patient. In various aspects, FIG. 5 illustrates an exemplary embodiment of user interfaces (UIs) that can be provided by the platform. In one aspect, FIG. 5 illustrates exemplary embodiments of an initial user interface (UI) 500 for a provider that can be provided by the platform, and FIG. 6 illustrate exemplary embodiments of an initial user interface (UI) 600 for patients that can be provided by the platform. Initial UIs can be used as an interface for a user to create and register a profile. An initial user interface can comprise various input fields and buttons to enable the user to provide data and choices to guide underlying user and services matching.

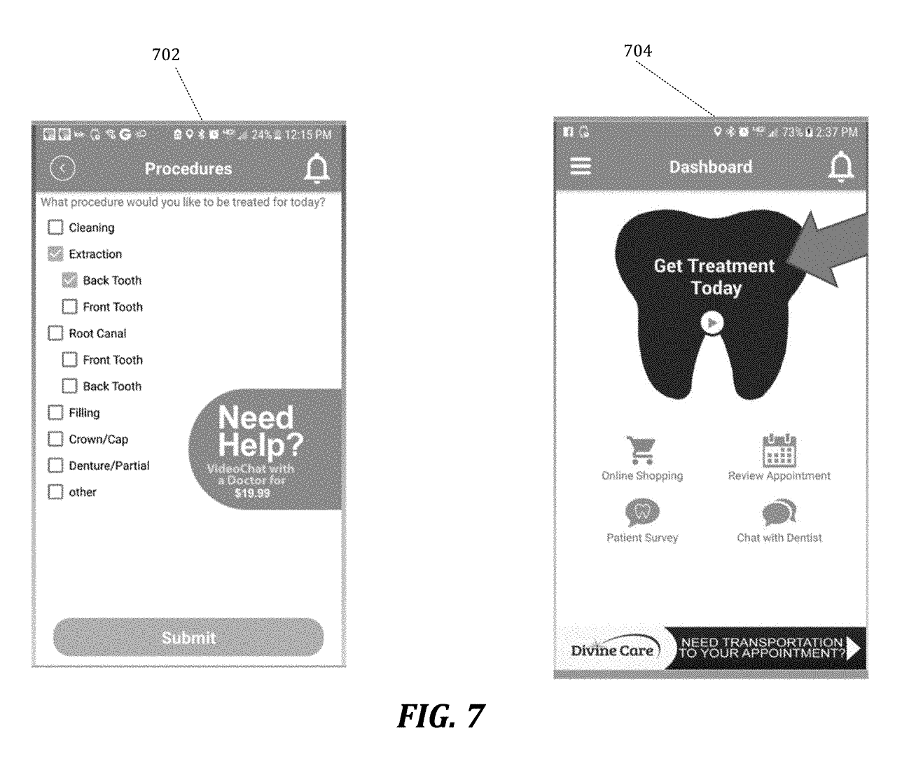

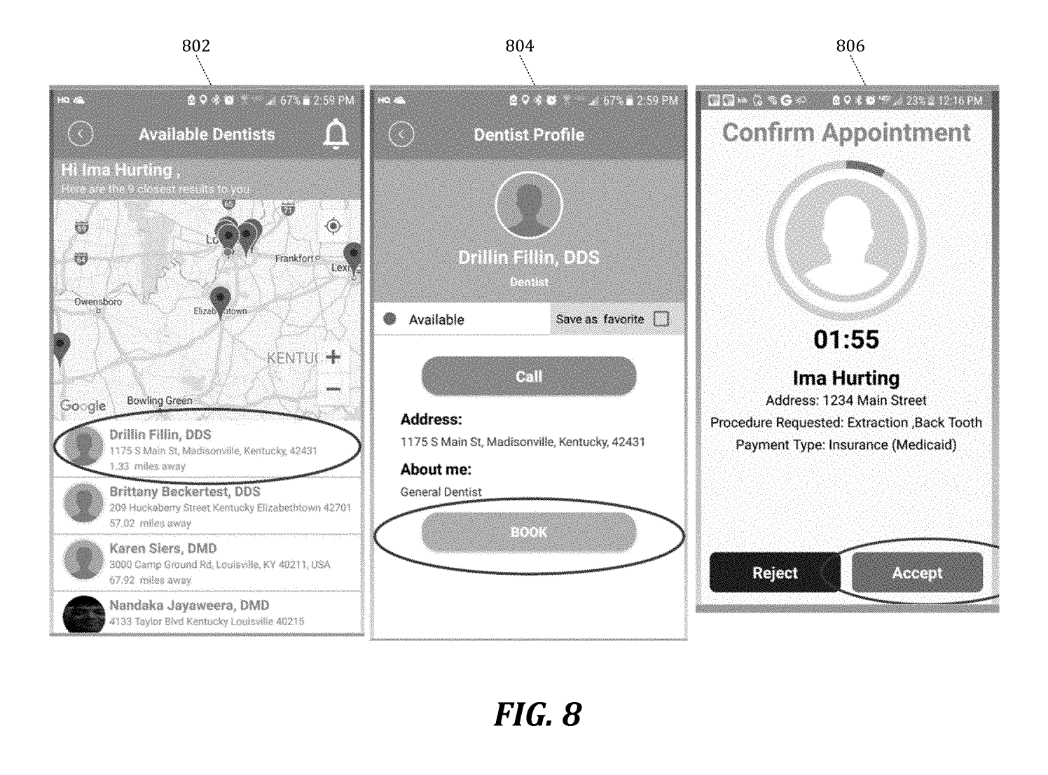

As shown in FIG. 7, the platform may provide patient a service UI 702, for a patient, where patient-users can select services and/or service criteria for finding matching service providers and services, and patient-user dashboard UI 704, for managing user account, requesting service appointments, communicating with other users/providers, and as further described herein. As shown in FIG. 8, patient profile data can be processed and analyzed with provider profile data to identify and display on UI 802, a set of matching providers and/or services on a map. The map displayed on the UI 802 can comprise providers' location pins can act as a window to their profile, providing provider specific information. Furthermore, the platform then allows the user to book an appointment with a provider on UI 804, such as by an appointment request, which can optionally be followed by an appointment confirmation on UI 806.

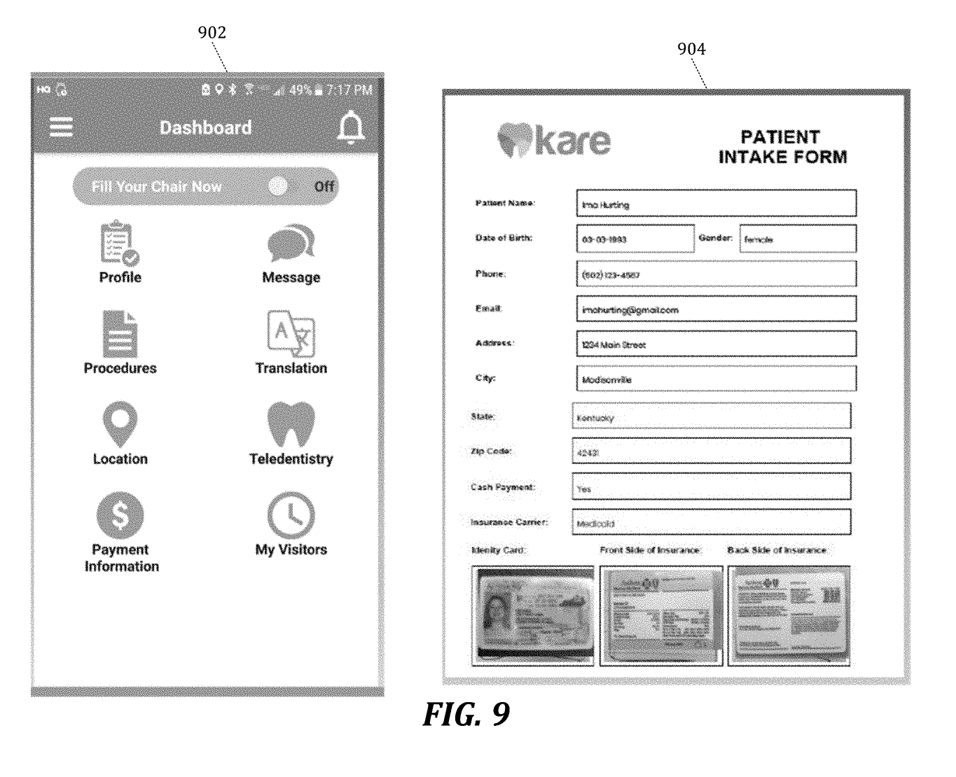

As shown in FIG. 9, the platform can by utilized to provide tele-dentistry services, for example, using the disclosed intraoral visualization devices and systems. For example, in exemplary aspects, the platform provides a method for visualization of a patient's mouth and/or teeth using a disclosed intraoral visualization device or system. In further aspects, the method may further comprise treatment of an area located in the mouth. In still further aspects, the treatment may be treatment of one or more teeth. FIG. 9 illustrates an exemplary embodiment of a provider UI 902 for tele-dentistry services that can be provided by the platform. Provider UI can be used as an interface for a provider to render tele-dentistry services to a user in connection with or independent of a disclosed intraoral visualization device. For example, the platform can be utilized to generate various forms and reports related to a patient user, such as a patient intake form 904, reports comprising image data captured using an intraoral visualization device during an examination. The forms and reports can be utilized by the provider, saved to the patient profile, and/or transmitted by the platform, for example, to a third party, such as an insurance company or third-party payer. In further aspects, the reports may comprise diagnostic reports that include image data and/or location data of the patient prior to treatment and/or treatment reports that include image data and/or location data of the patient after receiving treatment. In some aspects, a diagnostic report can be utilized and/or transmitted by the platform to a third party, such as an insurance company or third-party payer, to request authorization to render a dental service and/or treatment. The third party may utilize the report with image data to approve or deny an authorization request. To this end, the third party can use the image data to confirm that the requested services are medically warranted and/or use the location data, which can comprise GPS location data and/or user mobile device identification data, to confirm the identity of the patient user. In other aspects, a treatment report can be utilized and/or transmitted by the platform to a third party, such as an insurance company or third-party payer, to request payment for dental service and/or treatment rendered to a patient user. The third party may utilize the report, which may include image data and/or location related to the patient to approve or deny a payment request. To this end, the third party can use the image data to confirm that the services were rendered and use the location data, which can comprise GPS location data and/or user mobile device identification data, to confirm the identity of the patient user. The location data

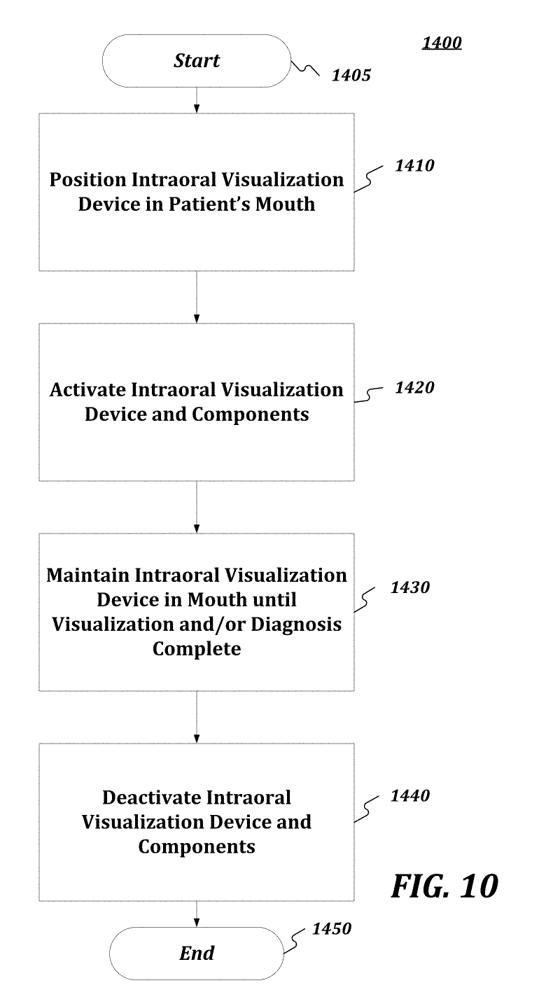

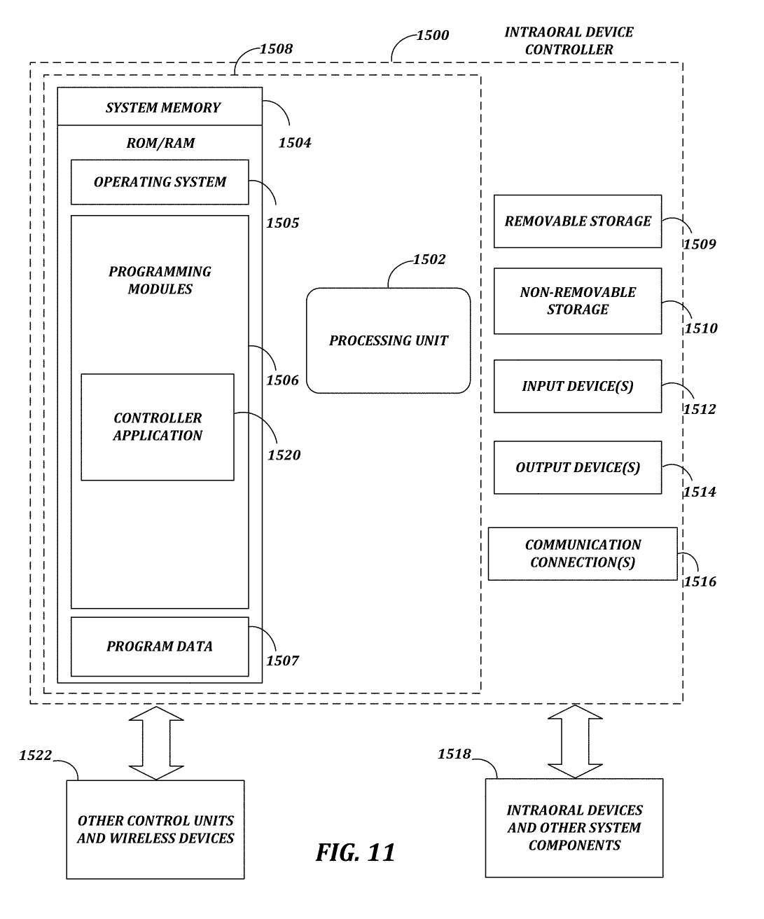

FIG. 10 is a flow chart setting forth the general stages involved in a method 1400 consistent with an embodiment of the disclosure for operating the disclosed intraoral visualization devices and systems. Method 1400 may be implemented using, at least in part, a controller 1500 (e.g., on-board computing device) as described in more detail below with respect to FIG. 11. Controller 1500 may comprise a controller for operating the imaging devices, lighting units, peripherals, and system components as well as well as performing other operational tasks, including, but not limited to, imaging and lighting control and parameters, and communication. As such, controller 1500 may be in operative configuration and communication with, for example, but not be limited to, imaging sensors, lighting units, activating switch, communication module, power module, various telemetry sensors, transceivers and antennas. As will be detailed with reference to FIG. 11, controller 1500 may comprise a remote communication module to enable remotely operation as described herein. In other embodiments, controller 1500 may be completely self-operating upon configuration.

Furthermore, although stages are disclosed with reference to controller 1500, it should be understood that a plurality of other components may enable the operation of method 1400, including, but not limited to, other computing components, mechanical components, environment properties (e.g., temperature), patient conditions, and the like. Further still, although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein.

Method 1400 may begin at starting block 1405 and proceed to stage 1410, where the intraoral visualization device may be placed or positioned in a mouth of a user or patient. From stage 1410, where the intraoral visualization device is positioned on the user's mouth, method 1400 may proceed to stage 1420 where the imaging devices, lighting units, and/or system components may be activated. The activation of components, though may be disclosed in a particular order for illustrative purposes, may occur in other arrangements. Upon activation, one or more lighting units may begin to illuminate and/or one or more imaging devices may begin to capture image data. In some embodiments where present, one or more projection elements may be activated to project assistive features onto the treatment surface.

In other aspects, an advantage of the invention can be that it allows a user to activate the intraoral visualization device components remotely from the device. In further aspects, a user does not have to pick up the intraoral visualization device to: activate its operation, to shut off operation, capture image data, and/or in embodiments that allow for adjustment, to adjust the illumination level. To this end, a user (e.g., dentist or dental operator) may activate or de-activate (and/or otherwise control operation of) the intraoral visualization devices by using a control unit, such as a wireless device or mobile device that is in operative communication with the intraoral visualization device. The wireless device may be a device that may be used for additional purposes other than use with the invention such as a mobile phone, tablet computer, notebook computer, desktop computer, etc. In an embodiment, the invention may provide a specialized wireless device for use with the invention. The specialized wireless device may include other uses, if its use is not limited to this particular embodiment of the invention.

As provided in more detail herein, the control unit used to control the intraoral visualization device may include an application or application software (an “app”) specifically created for such usage. Advantageously, the user may download and/or otherwise obtain the app from sources that supply apps such as independent developers and app stores. The app as used with embodiments of the invention communicates wirelessly, such as by using Bluetooth or the like technology.

In further aspects, controller 1500 (e.g., on-board computing-device) may automatically activate imaging devices and/or lighting elements instantly or after a set amount of time has passed since the launch. In other embodiments, activation may occur upon certain reading from on-board sensors (e.g., including, but not limited to, telemetry sensors deployed in the intraoral visualization devices). For example, activation of one or more imaging devices and/or lighting elements may be dependent on certain environmental factors and/or patient conditions such as, for example, oral temperature, blood pressure, patient diagnostic information, patient identifying information (e.g., fingerprint and/or facial recognition, device ID, device geolocation, etc.), and the like. In still further aspects, controller 1500 may be configured to trigger activation of various device components upon the satisfaction of certain pre-set conditions. Such conditions may be defined prior to activation.

From stage 1420, where the device components are activated, method 1400 may proceed to stage 1430, where the illumination effects may be maintained the site of the mouth for a time sufficient to perform visualization, diagnose and/or treat the site. From stage 1430, where the intraoral visualization device is used to perform a visualization, diagnosis, and/or treatment, method 1400 may proceed to stage 1440, where the device components are turned off. After stage 1440, method 1400 may end at stage 1450. In various aspects, methods of the present invention may comprise placing the device at a site in the mouth experiencing pain, discomfort, or may be placed above or proximal to such sites. In other aspects, the pain may be related to a dental cavity, infection or oral pathology. In further aspects, the intraoral visualization device may be allowed to act upon the patient for a time necessary to complete visualization, diagnosis and/or treatment which can be for a period of seconds up to a period of several minutes or more, or may be from greater than 0 seconds to about 120 minutes. If prolonged treatment is desired, the device may be mounted for a longer period. Once the desired outcome is reached, the intraoral visualization devices may be turned off and/or removed from the patient.

During all stages of the various methods, the intraoral visualization devices may be in operable communication with the user (e.g., dental operator) and/or patient via antenna or wireless communication component. The user may receive visualization data and/or various readings from the various device components. In some embodiments, the user may control the operation of the during use. For example, the user may be able to control the device components, including, but not limited to, imaging sensors, lighting units, activating switch, communication module, power module, various telemetry sensors, transceivers and antennas. In other embodiments, controller 1500 may be pre-configured with operational control instructions and/or data, for example, in connection with an established diagnostic and/or treatment protocol. Although operation has been described to be performed, in part, by a controller 1500, it should be understood that, in some embodiments, different operations may be performed by different networked elements in operative communication with controller 1500. Embodiments of the present disclosure may comprise a system having a memory storage and a processing unit. The processing unit may be coupled to the memory storage, wherein the processing unit is configured to perform the stages of method 1400.

FIG. 11 is a block diagram of a system including controller 1500. Consistent with an embodiment of the disclosure, the aforementioned memory storage and processing unit may be implemented in a computing device, such as controller 1500. Any suitable combination of hardware, software, or firmware may be used to implement the memory storage and processing unit. For example, the memory storage and processing unit may be implemented with controller 1500 or any of devices and components 1518, or any other control unit and wireless devices 1522, in combination with controller 1500. Other intraoral visualization devices and components 1518 may comprise, for example, but not be limited to, control mechanisms, imaging sensors, lighting units, activating switch, communication module, power module, various telemetry sensors, transceivers and antennas. The aforementioned system, device, and processors are examples and other systems, devices, and processors may comprise the aforementioned memory storage and processing unit, consistent with embodiments of the disclosure.

In further aspects, a system consistent with an embodiment of the disclosure may include a computing device, such as controller 1500. In a basic configuration, controller 1500 may include at least one processing unit 1502 and a system memory 1504. Depending on the configuration and type of computing device, system memory 1504 may comprise, but is not limited to, volatile (e.g. random-access memory (RAM)), non-volatile (e.g. read-only memory (ROM)), flash memory, or any combination. System memory 1504 may include operating system 1505, one or more programming modules 1506, and may include a program data 1507. Operating system 1505, for example, may be suitable for controlling controller 1500's operation. In one embodiment, programming modules 1506 may include controller application (“app”) 1520. Furthermore, embodiments of the disclosure may be practiced in conjunction with a graphics library, other operating systems, or any other application program and is not limited to any particular application or system. This basic configuration is illustrated in FIG. 11 by those components within a dashed line 1508.

Advantageously, the app may provide a user with information as well as be the user's interface to operating the embodiment of the invention. The app may include one or more graphic user interfaces (GUIs). Among the GUIs of the app may be a GUI allowing the user to pick which, if there is more than one, imaging device and/or lighting elements to activate, and to select (if available) one or more operating parameters or characteristics (such as intensity or lighting type) of the device(s). The user may be able to adjust such selections without having to deactivate the embodiment from a GUI of the app. The user may also use the app to turn on and turn off the device components. Another advantage of the app is that the app may present the user with a GUI that depicts the patient's mouth and/or teeth (or a generic mouth/teeth) and shows where the illumination and/or camera field of view are being applied. The GUI may include additional or other information relating to the image data being captured and may also present the user with information received from the device components, and or diagnostic information.

Controller 1500 may have additional features or functionality. For example, controller 1500 may also include additional data storage devices (removable and/or non-removable) such as, for example, magnetic disks, optical disks, or tape. Such additional storage is illustrated by a removable storage 1509 and a non-removable storage 1510. Computer storage media may include volatile and nonvolatile, removable and non-removable media implemented in any method or technology for storage of information, such as computer readable instructions, data structures, program modules, or other data. System memory 1504, removable storage 1509, and non-removable storage 1510 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by controller 1500. Any such computer storage media may be part of device 1500. Controller 1500 may also be operative with input device(s) 1512 such as a keyboard, a mouse, a pen, a sound input device, a touch input device, etc. Input device(s) 1512 may be used to, for example, manually access and program controller 1500. Output device(s) 1514 such as a display, speakers, a printer, etc. may also be included. The aforementioned devices are examples and others may be used.

Controller 1500 may also contain a communication connection 1516 that may allow device 1500 to communicate with other control units and wireless devices 1522 as well as intraoral visualization devices and other device components 1518 (e.g., transceivers, sensors, thermal elements), such as over an encrypted network in a distributed computing environment. Communication connection 1516 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, Bluetooth, radio frequency (RF), infrared, and other wireless media. The term computer readable media as used herein may include both storage media and communication media.

As stated above, a number of program modules and data files may be stored in system memory 1504, including operating system 1505. While executing on processing unit 1502, programming modules 1506 (e.g., intraoral visualization device controller application 1520) may perform processes including, for example, one or more of stages or portions of stages of method 1400 as described above. App 1520 may be configured to operate device components 1518 and receive instructions from, for example, communications connections module 1516. The aforementioned process is an example, and processing unit 1502 may perform other processes.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, multiprocessor systems, microprocessor-based or programmable consumer electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein can be different from the actual publication dates, which can require independent confirmation.

The patentable scope of the invention is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed:

1. An intraoral visualization system comprising:
at least one intraoral visualization device, each intraoral visualization device comprising:
a casing comprising a base and a head;
at least one imaging device contained within the head, the at least one imaging device configured to capture video data;
a plurality of lighting units contained within the head, each lighting unit comprising a discrete light source;
a communications module;
a power module; and
at least one switch in operative communication with and configured to control operation of at least one of: the at least one imaging device or the plurality of lighting units.

2. The system of claim 1, further comprising a controller configured to control operation of at least one of the following: the at least one imaging device, the communication module, at least one lighting unit of the plurality of lighting units, and the power module, based on at least one of the following:
a location of the apparatus,
an operating instruction received from the at least one switch,
an operating instruction received from the controller, and
an instruction received from the communications module.

3. The system of claim 1, wherein the controller is configured to communicate captured data to an access point via the communications module.

4. The system of claim 2, further comprising at least one telemetry sensor.

5. The system of claim 3, wherein the controller is configured to calculate telemetry data and communicate the telemetry data via the communications module along with captured video data.

6. The system of claim 3, wherein the at least one imaging device is configured to capture at least one of the following: teeth profile and oral pathologies.

7. The system of claim 6, wherein the power module is configured to be adapted to an external source for charging a battery associated with the power module.

8. The system of claim 6, wherein the at least one imaging device is a multi-depth focus camera configured to detect multiple light fields.

9. The system of claim 8, wherein the communications module utilizes at least one of the following: Wi-Fi, NFC, and Bluetooth technology.

10. The system of claim 8, further comprising a wireless device having application software configured to transmit instructions regarding the operation of the intraoral visualization device to the controller.

11. The system of claim 10, wherein the application software is operational to at least one of: present information regarding the operation of the at least one intraoral visualization device to a user via one or more graphic user interfaces on the wireless device, to receive the instructions from the user, and to transmit the instructions to the controller.

12. The system of claim 11, wherein at least one lighting unit comprises a white light source, and at least one lighting unit comprises a UV light source.

13. A method for providing tele-dentistry services to a subject, the method comprising the steps of:
a) using the system of claim 12 to capture video data from within a mouth of a subject; and
b) providing a dental service to the subject based at least on the captured video data.

14. The method of claim 13, further comprising the step of making a diagnosis based at least on the captured video data prior to the providing step; wherein the diagnosis comprises at least one oral pathology.

15. The method of claim 14, further comprising capturing location data associated with the subject; wherein the location data associated with the subject is used to verify an identity of the subject.

16. The method of claim 15, further comprising capturing video data from within the mouth of the subject after providing the dental service.

17. The method of claim 16, further comprising comparing post dental service video data with pre-dental service video data for confirmation dental service was rendered to subject.

18. The method of claim 17, wherein the confirmation is completed remotely by a third party.

19. The method of claim 18, wherein the third party is an insurance company or third-party payer; and wherein the confirmation is associated with a payment for the rendered dental service.

20. The method of claim 13, wherein the dental service comprises at least one of: a teeth cleaning, a tooth extraction, a root canal, a tooth filling, and a tooth crowning.
